(19) United States
(12) Patent Application Publication
(10) Pub. No.: US 2025/0121147 A1
(43) Pub. Date: Apr. 17, 2025

(54) ADJUSTABLE SECUREMENT DEVICE FOR ENDOTRACHEAL TUBE

(71) Applicant: Nuvance Health, Danbury, CT (US)

(72) Inventors: Veronica L. PASSARO, Danbury, CT (US); Kathleen BUDZIK, Ridgefield, CT (US); Dawn FURNISS, New Milford, CT (US); Karen MAURER, Stormville, NY (US); Sandra D. OMASTA, Danbury, CT (US); Christine RAYMOND, New Milford, CT (US); Alyssa SELINKA-NESS, New Milford, CT (US); Katherine SHAH, Southbury, CT (US); Jeffrey BARTLETT, Danbury, CT (US)

(21) Appl. No.: 18/974,121

(22) Filed: Dec. 9, 2024

Related U.S. Application Data
(63) Continuation of application No. PCT/US2023/024034, filed on May 31, 2023.
(60) Provisional application No. 63/347,942, filed on Jun. 1, 2022.
(60) Provisional application No. 63/358,035, filed on Jul. 1, 2022.

(51) Int. Cl. A61M 16/04

(52) U.S. Cl. CPC A61M 16/047

(57) ABSTRACT

The present disclosure generally relates to devices, and related methods of treatment using the same, for securing an endotracheal tube (commonly abbreviated as “ETT”) or other medical line to a patient, and more particularly to infants, babies in a neonatal unit, small children, etc.). The disclosure also relates to techniques and devices to remove the presence of unplanned extubations. In an exemplary embodiment, as taught in the present disclosure, a device may be used to secure an endotracheal tube to a small child or newborn infant such that the device may be configured to adjust the grip of the endotracheal tube to achieve optimal securement and stabilization of the endotracheal tube.

### CROSS-REFERENCE TO RELATED APPLICATIONS

This continuation application claims the benefit of and priority to International Patent Application No. PCT/US2023/024034, filed May 31, 2023, which claims priority to U.S. Provisional Patent Application No. 63/347,942, filed Jun. 1, 2022, and to U.S. Provisional Patent Application No. 63/358,035, filed Jul. 1, 2022, each of which are incorporated by reference in their entireties herein.

### TECHNICAL FIELD

The present disclosure generally relates to devices, and related methods of treatment using the same, for securing an endotracheal tube (commonly abbreviated as “ETT”) or other medical line to a patient, and more particularly to infants, babies in a neonatal unit, small children, etc.). The disclosure also relates to techniques and devices which prevent unplanned extubations. In an exemplary embodiment, as taught in the present disclosure, a device may be used to secure an endotracheal tube to a small child or newborn infant such that the device may be configured to adjust the grip of the endotracheal tube to achieve optimal securement and stabilization of the endotracheal tube.

### BACKGROUND

Endotracheal tubes are commonly used on medical patients, for example, to mechanically ventilated patients. Placement of an endotracheal tube into the throat of a patient is referred to as intubation. Failing to secure the endotracheal tube properly, however, can result in the migration of the tube within the throat of the patient. This may be referred to as an unplanned or unintended extubation. Such motion is undesirable since outward motion of the tube can result in the tube moving entirely out of the airway of the patient, eliminating its effectiveness, and potentially ventilating the patient's gastro-intestinal tract instead of their lungs. Inward motion of the tube is undesirable because it may eventually result in the tube moving down one of the patient's bronchi, preventing air from being ventilated to the other lung. This may quickly lead to the collapse of the unventilated lung. Even slight back and forth motion of the tube within the throat can result in tracheomalacia and ultimately in the weakening or collapse of the trachea, which may require permanent stenting to maintain an open airway. Migration may further result in a form of nosocomial pneumonia. This undesirable removal or movement of the endotracheal tube, alluded to above, may result in an unintended or accidental extubation. Unintended or accidental extubation is a serious medical emergency, which can have a dire outcome such as morbidity, permanent medical damage, or death.

With some patients, such as young children or neonates (e.g., newborn babies), the securement and stabilization of an endotracheal tube is critical, especially in the immediate days after birth to ensure that proper breathing and oxygen delivery is met. With such patients, current devices for gripping or securing the endotracheal tube to the baby's face or mouth may be sub-optimal due to a poorly designed or manufactured gripping device and/or owing to the baby's newborn physiology (i.e., newborn baby's skin composition and sensitivity to certain adhesives). In present endotracheal tube gripping devices commercially available, tabs and/or “lollipops” may become loose and lift from the young patient's face (e.g., the newborn baby's face). With some devices that utilize hook and loop fasteners (i.e., Velcro), the fasteners securing the endotracheal tube, if too loose, may cause the endotracheal tub to slip, and, if too tight, may not permit the suction catheter to pass. In such presently available tube gripping devices, if the endotracheal tube moves even slightly, it may cause an unplanned extubation, which may lead to the newborn baby losing critical oxygen supply or the delivery of other medical treatment through the tube. Certain existing devices may also block access to the oral cavity of the patient, resulting in difficulty in oral care which may lead to infection. Said infection may become particularly serious if it travels down the tube and into the lungs of the patient.

In order to avoid these undesirable consequences, it is common to secure the endotracheal tube in position upon the patient by means of adhesive tape. For instance, a medical practitioner may, after positioning the tube within the throat of the patient, wrap adhesive tape around the tube and tape it to the patient. Various devices are often used as adaptors for endotracheal tubes, and sometimes these devices are taped to the patient instead of directly taping the endotracheal tube to the patient. However, such devices do not allow for re-positioning or adjustment of the endotracheal tube within the device, and if tape or adhesives are used on the endotracheal tube, they are difficult to remove. With such devices or tape, adjustments of the endotracheal tube difficult and potentially dangerous for the patient.

Therefore, there is great need for an improved device, and related methods of treatment, to secure an endotracheal tube to a young patient (e.g., small children, newborn babies, etc.) to assist in the delivery of critical medical treatment, and for a device that maintains access to a young patient's mouth (e.g., a newborn baby's mouth). The present devices and methods meet these needs, and further provide for adjustable positioning of an endotracheal tube for secure placement in a patient.

### SUMMARY

Embodiments of the present disclosure relate to, among other things, devices, and related methods of treatment using the same, for secure and adjustable endotracheal tube stabilization in patients (e.g., small children, newborn babies, etc.). The devices, as described herein, and related methods of treatment using the devices, may be configured to be adjustable and may be configured to firmly grip an endotracheal tube to secure the tube to the patient's face, such that the device permits optimal delivery of oxygen or other medical treatment and removes the presence of unplanned extubations. An unplanned extubation may also be referred to as an unintended extubation or accidental extubation. In an exemplary embodiment, the devices may be positioned or otherwise secured to the young patient's face (e.g., newborn baby's face) using an adhesive securing means, and formed with one or more fastening mechanisms to permit securement and readjustment of the endotracheal tube. In some embodiments, the devices may be sterilizable or autoclavable for repeated use, or may be single-use/disposable.

In an embodiment, the present disclosure provides for a device for releasably securing a placed endotracheal tube, comprising: an elongated body having a first end and a second end, the elongated body having a first arm separated from a second arm along an axis of elongation of the elongated body; the first end including a hinge member connecting the first arm of the elongated body to the second arm of the elongated body; the second end including a releasable securement mechanism which releasably retains the first arm of the elongated body relative to the second arm of the elongated body; the releasable securement mechanism including a first member on the first arm of the elongated body and a second member on the second arm of the elongated body, the first member and second member releasably engaging in one or more closed positions to retain the first arm of the elongated body relative to the second arm of the elongated body; a circular aperture located on the elongated body at a position in-between the first and second ends, the aperture having a central axis generally perpendicular to the axis of elongation, and each of the first and second arms of the elongated body defining at least a portion of the circular aperture; wherein, in at least one closed position, the circular aperture is configured and dimensioned to engage with an outer surface of an endotracheal tube to secure the endotracheal tube relative to the elongated body to prevent movement of the endotracheal tube along the central axis of the aperture.

In an embodiment, the first member and second member of the releasable securement mechanism include a plurality of teeth which engage to retain the first arm of the elongated body relative to the second arm of the elongated body in one or more positions.

In an embodiment, the first member of the releasable securement mechanism includes a plurality of teeth and the second member of the releasable securement mechanism includes a plurality of corresponding grooves, wherein one or more teeth engage with one or more grooves to retain the first arm of the elongated body relative to the second arm of the elongated body in one or more positions.

In an embodiment, the first member and second member of the releasable securement mechanism include button closures which are eclipsed in a closed position and secured by an inserted button member.

In an embodiment, at least one outer surface of the device is generally arcuate.

In an embodiment, the device has an overall arcuate shape.

In an embodiment, the first and second ends include passages for an adhesive means to secure the device to the face of a patient.

In an embodiment, the passages have a central axis generally parallel with the central axis of the aperture.

In an embodiment, the present disclosure provides for a device for releasably securing a placed endotracheal tube, comprising: an elongated body having a first end and a second end, the elongated body having a first arm separated from a second arm along an axis of elongation of the elongated body; the first and second ends each including a passage for securing the device to a patient; a circular aperture located on the elongated body at a position in-between the first and second ends, the aperture having a central axis generally perpendicular to the axis of elongation, and each of the first and second arms of the elongated body defining at least a portion of the circular aperture; wherein the first arm and second arm are securable to engage the circular aperture with an outer surface of an endotracheal tube to secure the endotracheal tube relative to the elongated body, and wherein the aperture includes one or more retention features for engaging with the endotracheal tube to prevent movement of the endotracheal tube along the central axis of the aperture.

In an embodiment, the one or more retention features include ridges on an inner surface of the aperture.

In an embodiment, the ridges are aligned substantially parallel with the central axis.

In an embodiment, the ridges are aligned substantially perpendicular to the central axis to define ridges concentric with the aperture.

In an embodiment, the one or more retention features include one or more raised features.

In an embodiment, the one or more retention features include a rubberized or non-slip coating.

In an embodiment, the present disclosure provides for a device for releasably securing a placed endotracheal tube, comprising: an elongated body having a first end and a second end, the elongated body having a first arm separated from a second arm along an axis of elongation of the elongated body, the elongated body having at least one outer surface which is generally arcuate; the first end including a hinge member connecting the first arm of the elongated body to the second arm of the elongated body; the second end including a releasable securement mechanism which releasably retains the first arm of the elongated body relative to the second arm of the elongated body; the releasable securement mechanism including a first member on the first arm of the elongated body and a second member on the second arm of the elongated body, the first member and second member releasably engaging in one or more closed positions to retain the first arm of the elongated body relative to the second arm of the elongated body; a circular aperture located on the elongated body at a position in-between the first and second ends, the aperture having a central axis generally perpendicular to the axis of elongation, and each of the first and second arms of the elongated body defining at least a portion of the circular aperture; wherein, in at least one closed position, the circular aperture is configured and dimensioned to engage with an outer surface of an endotracheal tube to secure the endotracheal tube relative to the elongated body to prevent movement of the endotracheal tube along the central axis of the aperture.

In an embodiment, the device has an overall arcuate shape.

In an embodiment, the at least one outer surface is generally arcuate along the axis of elongation.

In an embodiment, the at least one outer surface is arcuate to conform to a mouth region of a patient's face.

In an embodiment, the angular curvature of the generally arcuate outer surface is from about 30 degrees to about 1 degree.

In an embodiment, the axis of elongation is from about 3 cm to about 10 cm.

In an embodiment, the axis of elongation is from about 5 cm to about 7 cm.

In an embodiment, a curvature of the generally arcuate outer surface is generally circular and ranges from about 0.04 rad/mm to about 0.001 rad/mm.

In an embodiment, the curvature of the generally arcuate outer surface is from about 0.02 rad/mm to about 0.005 rad/mm.

In an embodiment, a chord of the generally circular curvature is from about 2 cm to about 8 cm.

In an embodiment, an arc length of the generally circular curvature is from about 3 cm to about 10 cm.

In an embodiment, the disclosure generally provides for a method of securing an endotracheal tube to a young patient in need thereof, comprising: placing an endotracheal tube in the patient; securing an endotracheal tube securement device to the endotracheal tube at a position which maintains a desired depth of insertion of the endotracheal tube with the securement device placed against the mouth of the patient; and securing the endotracheal tube to the mouth of the patient using an adhesive securement means.

In an embodiment, the adhesive securement means is cloth tape.

### BRIEF DESCRIPTION OF THE DRAWINGS

The accompanying drawings, which are incorporated in and constitute a part of this specification, illustrate exemplary embodiments of the present disclosure and together with the description, serve to explain the principles of the disclosure.

FIG. 1 depicts a perspective view of an exemplary clamping device equipped with an endotracheal tube, according to an aspect of the present disclosure.

FIG. 2 depicts a top view of the exemplary clamping device of FIG. 1, wherein the clamping device is in a closed or clamped position to secure the endotracheal tube, according to an aspect of the present disclosure.

FIG. 3 depicts a top view of the exemplary clamping device of FIG. 1, wherein the clamping device is in an open or unclamped position to release the endotracheal tube, according to an aspect of the present disclosure.

FIGS. 4A-4B depict exemplary releasable securement mechanisms for the devices as described herein. FIG. 4A depicts an exemplary toothed mechanism and FIG. 4B depicts an exemplary tooth/groove mechanism.

FIG. 5 depicts an exemplary embodiment of a button closure.

FIG. 6 depicts an exemplary curved or arcuate device of the present disclosure.

FIG. 7 depicts an exemplary embodiment on a face of an infant patient with an endotracheal tube (ETT) insertion direction shown.

FIGS. 8A-8B depict alternative exemplary releasable securement mechanisms.

FIG. 8A depicts a tongue-and-groove mechanism and FIG. 8B depicts an overlapping mechanism.

### DETAILED DESCRIPTION

### Definitions

The abbreviation ETT as used herein means “endotracheal tube.”

The term “extubation” as used herein means removal of the endotracheal tube from the patient's mouth such that the patient is no longer being intubated.

The term “releasably adjustable” as use herein means that the exemplary clamping device, described herein, may be able to be opened and closed repeatedly to allow for re-positioning of the endotracheal tube to ensure no accidental or undesirable extubation(s) occur. The “releasably” may refer to the clamping or unclamping open/closed features of the clamping device, and the “adjustable” may refer to the positioning of the endotracheal tube along any portion of the clamping device's arms, and more particularly along a central axis of an aperture configured to secure an endotracheal tube.

The term “adhesive means” as used herein refers to various adhesives which may be used to secure the devices of the present disclosure to the face of a patient. Adhesive means include, but are not limited to, tape, cloth tape, woven tape, adhesive textile, double-sided tape, single-sided tape, medical tape, hypoallergenic tape, waterproof tape, stretch tape, elastic tape, foam tape, or any other tape or adhesive sufficient to secure the present devices. Adhesive means may be used in conjunction with an opening or passage in a device to secure the device to a mouth region of a patient's face.

### Overview

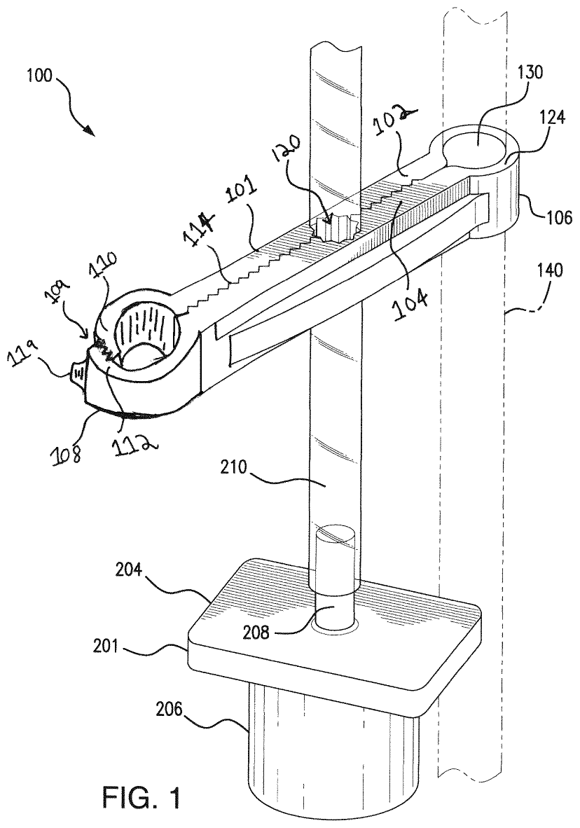

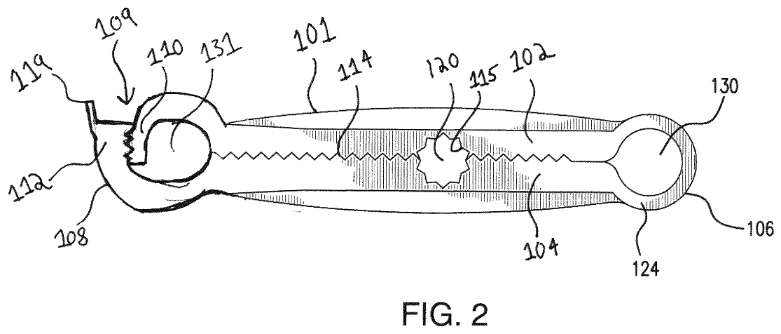

The present disclosure relates to devices for securement of an endotracheal tube to a young patient's face or mouth (e.g., small child, newborn baby, etc.) that is adjustable, and methods for treatment using the same. In FIG. 1, and according to an exemplary embodiment, a clamping device system 100 is shown, wherein the system 100 includes a clamping device 101 with an endotracheal tube 210 installed through an aperture 120 of the clamping device 101. FIG. 2 displays the clamping device 101 of FIG. 1 in a closed position, without an endotracheal tube installed. The clamping device 101 may be formed from a single piece of plastic, or similar material and may be disposable. The clamping device 101 may be manufactured using additive manufacturing (e.g., 3D printing) or similar methods. In an exemplary embodiment, the clamping device 101 is about 6 cm long×5 mm wide. In some embodiments, the clamping device 101 may be curved or substantially non-planar, such that the clamping device is configured to be seated or located over a mouth of a patient (e.g., neonate baby).

In FIG. 1, the endotracheal tube 210 is shown in an installed configuration within the clamping device 101. The endotracheal tube 210 may be sold separately from the clamping device 101 or together with the clamping device 101. The endotracheal tube 210 may include a base section 201 having a top face 204, as shown in FIG. 1. From the top face 204 of the base section 201, a tube-shaped member 208 may extend from a center of the top face 204. An endotracheal tube 210 may be fluidly connected to the tube-shaped member 208 such that when oxygen (or other medical supplement used when intubating a patient for medical care) is pumped through a fluidly connected endotracheal tube adaptor 206, the oxygen may flow from adaptor section 206, into tube-shaped section 208, and into the endotracheal tube 210. The endotracheal tube 210 may be formed from medical grade plastic or similar material. The endotracheal tube 210 may be configured and dimensioned in a plurality of cross-sectional dimensions (e.g., diameters) such that the tube 210 may be inserted within the aperture 120 of the clamping device 101. It can be appreciated that the clamping device 101 may be used with the endotracheal tube 210 shown and described herein or alternatively the clamping device 101 may be used by any suitable commercially available endotracheal tube in the market. The clamping device 101 may, for example, be used with endotracheal tubes designed specifically for neonates or young adolescents. As will be described in greater detail, one or more passages or openings (e.g. passage 130) may generally provide for securing the device using an adhesive means 140 such as tape.

In FIG. 2, the clamping device 101 is shown in the closed, clamped position, and without the endotracheal tube 210. The clamping device 101 may be formed as an integral piece, unitary piece, from one or more materials (e.g., medical-grade plastic). The clamping device 101 may include two, opposed clamping arms 102 and 104 that may be clamped shut to hold or secure an endotracheal tube 210 within the aperture 120. The opposed clamping arms may be generally described as separate arms, portions, or halves split along an axis of elongation of the device 101, where the axis of elongation is oriented in the major or longest dimension of the device.

As shown in FIG. 2, a plurality of serrations 114 may be formed on an inner surface of the two, opposed clamping arms 102 and 104, such that when the two clamping arms 102 and 104 of the clamping device 101 are brought together, the serrations 114 close together to create a toothed-like grip. The serrations 114, if present, may assist in preventing movement of the arms in a closed state. In an alternative embodiment, the device does not have serrations 114. Engagement features 115 may also be formed within the aperture 120, in an exemplary embodiment, so as to secure or grip an endotracheal tube, as shown in FIG. 1. Engagement features 115 may include serrations, ridges, or other raised features that grip the endotracheal tube in a closed configuration. Ridges and serrations may be aligned in a concentric direction surrounding the aperture, in a direction parallel with the axis of the inserted endotracheal tube, diagonally, or in any other direction. Engagement features 115 may also include patterned or random raised features (for example, bumps), or alternatively a non-slip coating such as a rubberized coating. In an exemplary embodiment, the engagement features 115 may be substantially the size and shape at the plurality of serrations 114. In another exemplary embodiment, the engagement features 115 within the aperture may be different in size and shape from the serrations 114. The engagement features 115 may be formed such that they are not sharp enough to tear, rupture, or displace or otherwise disturb an installed endotracheal tube within the clamping device 101; however, they would be formed to optimally hold or secure the tube to prevent any slippage of the endotracheal tube upon installation in a young patient (e.g., neonate, small child, etc.).

In some exemplary embodiments, the clamping device 101 may include the serrations 114 and/or 115 along both arms and within the aperture. In an exemplary embodiment of the device 101, there may be six serrations per cm throughout the device (e.g., density of serrations).

A first end 106 of the clamping device 101 may include a closed, integrally formed hinge member 124. In an embodiment, the end 106 may be formed as substantially an “O-shaped” member, such that hinge member 124 is flexible to allow the first and second halves or arms of the device to open and close relative to one another. The end 106 may be formed integrally with the rest of the clamping device 101 such that the clamping arms 102 and 104 meet together at the end 106.

While an “O-shaped” end 106 is exemplary, the end 106 and hinge member 124 may take any shape or form so long as the end 106 functions as a hinge, and in some embodiments generally provides for a passage 130, alternatively referred to as an opening for an adhesive means 140. In an embodiment, the hinge member 124 defines passage 130. In an alternative embodiment, a passage 130 is provided or disposed on an exterior portion of the end 106 such that the hinge member 124 does not define the passage 130. In an alternative embodiment, end 106 includes an adaptor or other means for coupling with an adhesive means 140.

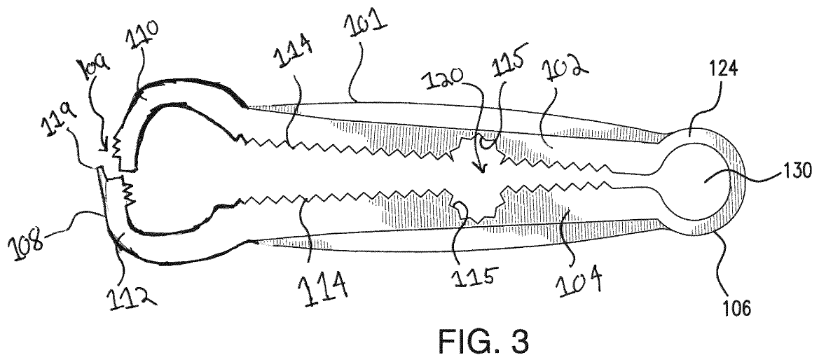

The clamping device 101 may also include an opposed second end 108, as shown in FIGS. 2 and 3, that may be releasably opened (see FIG. 3) and closed (see FIG. 2) using a locking mechanism 109, alternatively referred to as a releasable securement mechanism. In an exemplary embodiment, the locking mechanism 109 may include locking members 110 and 112. The locking member 110 is disposed on clamping arm 102 and the locking member 112 is disposed on clamping arm 104. The locking members 110 and 112 are integrally formed within their respective clamping arms 102 and 104, respectively. During operation of the clamping device 101, when a user (e.g., nurse, doctor, or individual charged with the management and/or installation of the endotracheal tube on a patient) closes or brings the two clamping arms 102 and 104 together, the clamping device 101 may be locked or fastened using the locking mechanism 109 at the end 108. That is, the arms may be retained in one or more positions relative to one another such that they will not move unless tightened further or released.

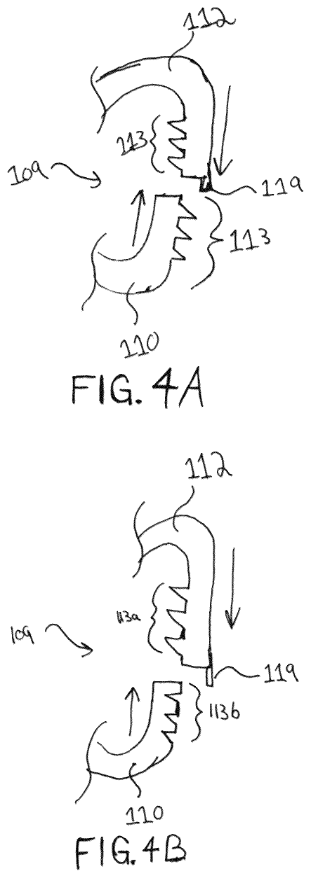

Exemplary locking mechanisms 109 are depicted in FIGS. 4A and 4B. In an embodiment, as depicted in FIG. 4A, each of the locking members 110 and 112 include teeth 113 which may be evenly spaced and which may engage when they are moved past one another during closure of locking member 110 with locking member 112. In an alternative embodiment, as depicted in FIG. 4B, one locking member (110 or 112) includes teeth 113a and the other locking member includes corresponding grooves which are configured and spaced to engage with the teeth. Generally, when an endotracheal tube 210 is inserted into aperture 120 of the device, some pressure will need to be applied to secure locking members 110 and 112 by teeth 113 or by teeth/grooves 113a/113b, and the teeth or teeth/grooves will generally remain engaged until the mechanism is released. The locking mechanism 109 is releasable and generally includes a means for disengaging the locking members 110 and 112 to separate the arms 102 and 104 of the device. In an embodiment, the means for disengaging is a pull tab 119 which allows locking member 112 to be pulled away from locking member 110 until their teeth 113 or teeth/grooves 113a/113b disengage to allow the arms 102 and 104 of the device to separate.

In an embodiment, the teeth 113 or teeth/grooves 113a/113b may take any shape or form so long as they hold the device 101 in a closed position to secure the endotracheal tube 210. In an embodiment, there are a plurality of teeth 113 or teeth/grooves 113a/113b so that the locking members 110 and 112 may be closed in a plurality of different positions so as to apply various gripping pressure on the endotracheal tube. In an embodiment, the locking members 110 and 112 each contain between 2 to 8 teeth, or 2 to 8 teeth and 2 to 8 corresponding grooves. In various alternative embodiments, teeth and/or grooves may be disposed on either or both sides of the locking members 110 and 112 provided that the device is configured and dimensioned such that the teeth 113 or teeth/grooves 113a/113b of the locking members 110 and 112 will interfere so as to releasably lock the device in a closed position to secure an endotracheal tube 210. In a closed position, the locking mechanism may provide a passage 131 for an adhesive means 140, such that each end of the device contains such a passage for securing the device to the face of a patient. It can be appreciated that the locking mechanisms of FIG. 4A and FIG. 4B are exemplary and any suitable, releasable locking mechanism is contemplated.

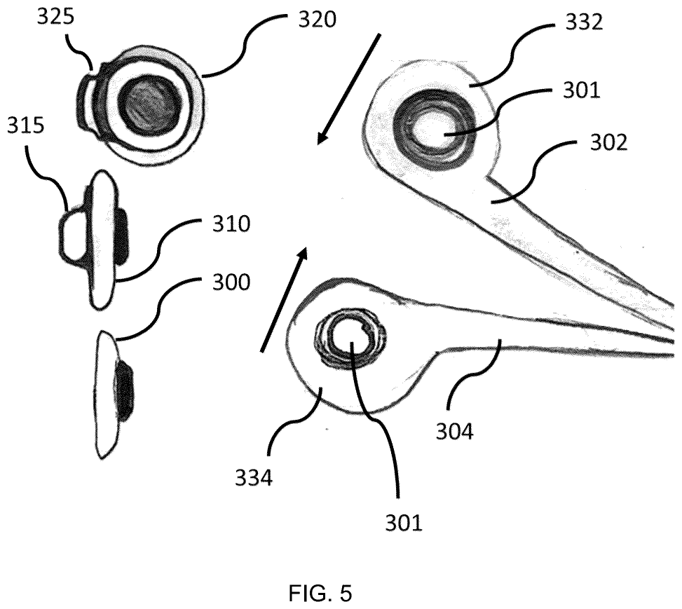

In some exemplary embodiments, the clamping device 101 may be formed with alternative locking mechanisms other than the locking mechanism 109 alluded to above. For example, in an exemplary embodiment the “closure” or locking mechanism to secure the clamping arms 102 and 104 may be formed with an “O” closure with a “button” that may be depressed in order to have opposing “O” click in place over it, as shown in FIG. 5. The button 300 generally may be inserted into overlapping or eclipsed button closures 301 when the ends 332 and 334 of the device are closed together. In a closed position, the arms 302 and 304 will be pulled together so as to secure the endotracheal tube in the aperture of the device as described herein. The button may be a plain button closure 300, or may alternatively be a button 310 with a loop 315 on the back, or may alternatively be a button 320 with a loop 325 located at a side. A loop 315 or 325 is generally useful for providing a passage for a securement means as described herein, such as a cloth tape. The button closure is releasable if the button is removed from the eclipsed closures 301.

In yet another exemplary embodiment, the “O” shapes, described above, may be sized in a compatible manner in order to slide together and click in place with or without a flexible tooth/groove closure to hold both “O” shapes together. In yet another exemplary embodiment, the ends may have notches or holes for using wire or an adhesive means to secure the ends in a closed position. In yet another exemplary embodiment, the ends may interfere or be closed together in any manner.

In an exemplary embodiment, the device may have a curved profile, of the device may be generally arcuate. Alternatively, at least one outer surface may be generally arcuate or curved, meaning that in addition to the required curved outer surface, any other portion of the device, including the entire device, may be curved. The at least one outer surface is generally curved so as to conform with a shape of a mouth region of a patient's face for comfort and improved stability of the device in a secured state.

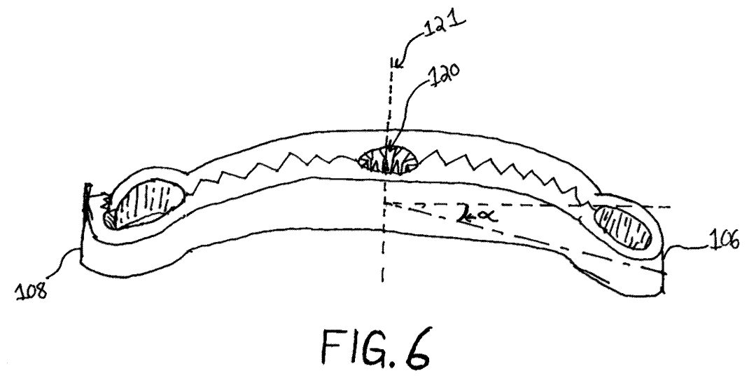

An exemplary arcuate or curved device is show in in FIG. 6. The curvature of the at least one face, or in this case of the entire device, is generally along an axis 121 of the aperture 120/inserted endotracheal tube 210. That is, if the endotracheal tube 210 is inserted from the top of FIG. 6 into the top of the aperture, the ends 106 and 108 will generally curve away from the top of the aperture. The curvature may be alternatively described as generally parallel with the axis of elongation of the device, because the longest axis (i.e. axis of elongation) of the device will become longer when the device is curved compared to a straight or linear device. It is intended that the axis of elongation can be a curved axis when the device is arcuate or curved.

The curvature of the device may be defined in various manners. For example, as shown in FIG. 6, the device may be placed on a vertical axis 121 which passes through the center of the aperture with the ends 106 and 108 being generally horizontal. An x-axis may be placed at a vertical center of the aperture and a diagonal axis may be placed to bisect an end (end 106 in this case). An angle α may then be defined as the angle between the x-axis and the diagonal axis to provide an “angular curvature”. In an embodiment, the angular curvature of the device (i.e. angle α) is about 1 degree to about 30 degrees. In an embodiment, angle α is about 5 degrees to about 20 degrees. In an embodiment, angle α is about 5 degrees to about 15 degrees.

In alternative embodiments, the curvature may also be defined by approximation of the at least one arcuate surface (or entire device in some cases) to a circle. For a circle, the curvature may be defined by the inverse of the radius (e.g. in mm) with units of rad/mm. In an embodiment, the curvature is from about 0.04 rad/mm to about 0.001 rad/mm. In an alternative embodiment, the curvature is from about 0.02 rad/mm to about 0.005 rad/mm. In some alternative embodiments where the curvature is approximated by a circle, the dimensions may be alternatively or additionally defined by a chord of the circle. In an embodiment, the chord is from about 2 cm to about 8 cm, or from about 4 cm to about 7 cm. Per the standard geometric definition, a “chord” is a straight line segment whose endpoints both lie on a circular arc.

In alternative embodiments, the locking mechanism 109 may include any features useful for retaining locking members 110 and 112 in a closed state for device 101 to secure an endotracheal tube. For example, in an embodiment, a tongue-and-groove type of retaining feature 113c may be included. The tongue may be retained in the groove by friction or by one or more other features in the tongue or groove which releasably lock the locking members 110 and 112 in a closed state. In another embodiment, an overlapping type of retaining feature 113d may be included. The overlapping features may be retained by tension from, for example, locking member 110 being stretched outward for its overlapping feature to overlap with the corresponding feature on locking member 112. Alternatively, the overlapping retaining feature(s) 113d may include one or more additional features which releasably lock the locking members 110 and 112 in a closed state. In an alternative embodiment, overlapping feature 113d may be alternatively or additionally secured in a closed state with an adhesive means such as tape. It can be appreciated that various adaptations of the locking mechanisms 109 are within the scope of the present disclosure.

In an exemplary embodiment, the endotracheal tube that is to be secured or held by the clamping device 101, taught herein, may vary in size (e.g., diameter of the endotracheal tube may change) depending on the weight and/or body mass index of the patient (e.g., larger or smaller newborn babies). The diameter of the aperture 120, for example, may correspond with the following patient body weights:
4.0 mm for >3 kg;
3.5 mm for 2-3 kg;
3.0 mm for 1-2 kg; or
2.5 mm for <1 kg.

In an exemplary embodiment, each size of the clamping device 101 may be formed using a different color, and each size may be slightly bigger to accommodate a larger infant.

In some exemplary embodiments, the clamping device 101 may be manufactured in four (4) different sizes to accommodate four (4) different neonatal size groups. The table below is an example of the four different neonatal size groups:

| Sizes | Length | Color | ETT Hole Size |
|---|---|---|---|
| <1 kg, <28 weeks | 5.5 cm | Neon Pink | 2.5 mm |
| 1-2 kg, 28-34 weeks | 6.0 cm | Pastel Green | 3.0 mm |
| 2-3 kg, 34-38 weeks | 6.5 cm | Pastel Blue | 3.5 mm |
| 3-4 kg, >38 weeks | 7.0 cm | Pastel Yellow | 4.0 mm |

In some embodiments of the clamping device 101, for example in the 5.5 cm version of the clamping device 101 and in the 6.0 cm version of the clamping device 101, these two versions may have more than one ETT hole size choice, each with another color. For example, see table below:

| | |
|---|---|
| 5.5 cm Length | 6.0 cm Length |
| 2.5 mm ETT Neon Pink | 2.5 mm ETT Neon Green |
| 3.0 mm ETT Pastel Pink | 3.0 mm ETT Pastel Green |

In some exemplary embodiments of the clamping device 101, a skin protectant may be applied, either during manufacture of the clamping device 101 or after the clamping device 101 is made. The skin protectant may be applied to any surface of the clamping device that may be in contact with the patient's skin or mouth. For example, the skin protected described herein may be applied to one or more of the arms 102 or 104 that may be in contact with a patient's face or mouth (e.g., a neonate's face or mouth). In an exemplary embodiment, the skin protectant may be a hydrocolloid or other skin protectant material and may be used only on the underside of the device. In some exemplary embodiments, the skin protectant may be replaced as needed (for example, by a NICU nurse or similar practitioner), or an extra piece may be pre-cut and included with the clamping device 101.

In an exemplary embodiment, the clamping device 101 may include two skin protectant strips (e.g., hydrocolloid) adhered to a bottom, curved side of the device 101 that would be closest to the patient's face and on either side of the ETT. The strips may fall off due to wear, secretions, etc. and a replacement pair of hydrocolloid strips may be included with the clamping device 101.

### Method of Treatment Using Securement

In some exemplary embodiments of the clamping device 101, the clamping device 101 may be used utilizing one or more of the steps below:
1—Cleanse a patient's face well and dry. Patient may include a neonate baby or young adolescent.
2—Place hydrocolloid barrier on cheeks of the patient.
3—Apply benzoin to barrier on cheeks of the patient.
4—Cut an adhesive means, such as a cloth tape, into strips 2-3 inches long and ½-1 inch wide depending on size of infant. Fold tabs at each end.
5—Split the tapes lengthwise down the center so each strip resembles a “Y.”
6—Attach ETT securement device around ETT, positioned to rest just above upper lip.
7—Place one piece of tape on hydrocolloid with split side facing ETT. Bifurcation should be at corner of the mouth of the patient. Wrap the lower end of the “Y” around the device and the upper end of the “Y” around the ETT above the device.
8—Repeat this process (steps 1-7 above) on the other side of the patient's mouth (e.g., infant's mouth). Alternate the upper and lower tapes so it is the opposite of taping on the other side. (upper tape around the clamp, and lower tape around the ETT).

In some exemplary alternative embodiments, the securement may be with hydrocolloid tabs with securement loops made of adhesive that will be looped through the passages or openings 130 and 131 defined at ends 106 and 108. One side can be replaced without replacing the whole device. Tabs with adhesive loops may be included with device.

In a preferred embodiment of the clamping device 101 may include the following features: a closure that is “O” shaped with tooth/groove clasp, device 101 may be 4 sizes with the 5.5 cm and 6.0 cm length each made with 2.5 and 3.0 mm holes, skin protectant can be replaced, and securement is with tape as described previously. Curvature and serrations as described previously.

In some embodiments of the clamping device 101, the following versions may be preferred. The preferred versions may include:
1. “O” closure/4 sizes with added hole sizes/replaceable skin protectant/tape secured
2. “O” closure/4 sizes with only 4 hole sizes/replaceable skin protectant/tape secured
3. “O” closure/4 sizes with added hole sizes/fixed skin protectant/tape secured
4. “O” closure/4 sizes with only 4 hole sizes/fixed skin protectant/tape secured
5. “O” closure/4 sizes with added hole sizes/replaceable skin protectant/tab secured
6. “O” closure/4 sizes with only 4 hole sizes/fixed skin protectant/tab secured
7. Button closure and any combination of the other elements

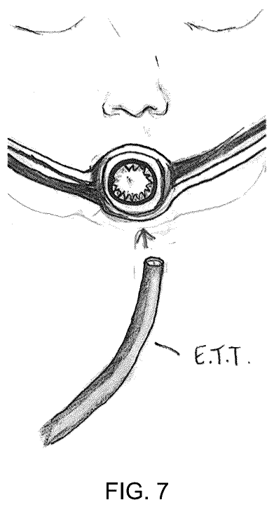

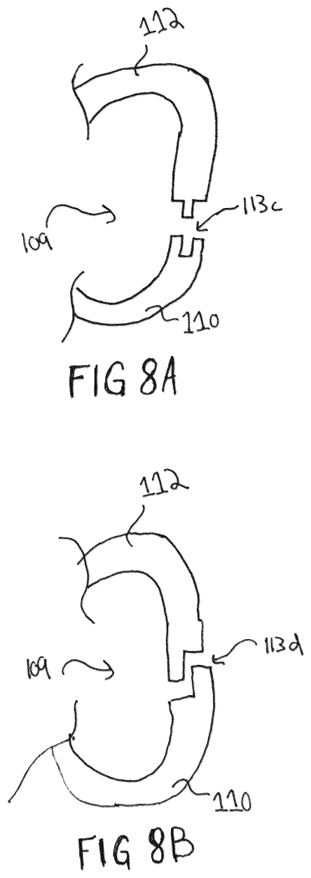

FIG. 7 depicts an exemplary embodiment shown on a face of an infant and is for illustrative purposes only. The device is generally curved along the face of the infant (for curved embodiments) with the aperture of the device facing away from the face of the patient. The endotracheal tube, when placed in the patient, will be secured in the aperture of the device (here shown with the tube removed for illustration).

While principles of the present disclosure are described herein with reference to illustrative embodiments for particular applications, it should be understood that the disclosure is not limited thereto. Those having ordinary skill in the art and access to the teachings provided herein will recognize additional modifications, applications, embodiments, and substitution of equivalents all fall within the scope of the embodiments described herein. Accordingly, the invention is not to be considered as limited by the foregoing description.

What is claimed is:

1. A device for releasably securing a placed endotracheal tube, comprising:
an elongated body having a first end and a second end, the elongated body having a first arm separated from a second arm along an axis of elongation of the elongated body;
the first end including a hinge member connecting the first arm of the elongated body to the second arm of the elongated body;
the second end including a releasable securement mechanism which releasably retains the first arm of the elongated body relative to the second arm of the elongated body;
the releasable securement mechanism including a first member on the first arm of the elongated body and a second member on the second arm of the elongated body, the first member and second member releasably engaging in one or more closed positions to retain the first arm of the elongated body relative to the second arm of the elongated body;
a circular aperture located on the elongated body at a position in-between the first and second ends, the aperture having a central axis generally perpendicular to the axis of elongation, and each of the first and second arms of the elongated body defining at least a portion of the circular aperture;
wherein, in at least one closed position, the circular aperture is configured and dimensioned to engage with an outer surface of an endotracheal tube to secure the endotracheal tube relative to the elongated body to prevent movement of the endotracheal tube along the central axis of the aperture.

2. The device of claim 1, wherein the first member and second member of the releasable securement mechanism include a plurality of teeth which engage to retain the first arm of the elongated body relative to the second arm of the elongated body in one or more positions.

3. The device of claim 1, wherein the first member of the releasable securement mechanism includes a plurality of teeth and the second member of the releasable securement mechanism includes a plurality of corresponding grooves, wherein one or more teeth engage with one or more grooves to retain the first arm of the elongated body relative to the second arm of the elongated body in one or more positions.

4. The device of claim 1, wherein the first member and second member of the releasable securement mechanism include button closures which are eclipsed in a closed position and secured by an inserted button member.

5. The device of claim 1, wherein at least one outer surface of the device is generally arcuate.

6. The device of claim 5, wherein the device has an overall arcuate shape.

7. The device of claim 1, wherein the first and second ends include passages for an adhesive means to secure the device to the face of a patient.

8. The device of claim 7, wherein the passages have a central axis generally parallel with the central axis of the aperture.

9. A device for releasably securing a placed endotracheal tube, comprising:
an elongated body having a first end and a second end, the elongated body having a first arm separated from a second arm along an axis of elongation of the elongated body;
the first and second ends each including a passage for securing the device to a patient;
a circular aperture located on the elongated body at a position in-between the first and second ends, the aperture having a central axis generally perpendicular to the axis of elongation, and each of the first and second arms of the elongated body defining at least a portion of the circular aperture;
wherein the first arm and second arm are securable to engage the circular aperture with an outer surface of an endotracheal tube to secure the endotracheal tube relative to the elongated body, and
wherein the aperture includes one or more retention features for engaging with the endotracheal tube to prevent movement of the endotracheal tube along the central axis of the aperture.

10. The device of claim 9, wherein the one or more retention features include ridges on an inner surface of the aperture.

11. The device of claim 10, wherein the ridges are aligned substantially parallel with the central axis.

12. The device of claim 10, wherein the ridges are aligned substantially perpendicular to the central axis to define ridges concentric with the aperture.

13. The device of claim 9, wherein the one or more retention features include one or more raised features.

14. The device of claim 9 wherein the one or more retention features include a rubberized or non-slip coating.

15. A device for releasably securing a placed endotracheal tube, comprising:
an elongated body having a first end and a second end, the elongated body having a first arm separated from a second arm along an axis of elongation of the elongated body, the elongated body having at least one outer surface which is generally arcuate;
the first end including a hinge member connecting the first arm of the elongated body to the second arm of the elongated body;
the second end including a releasable securement mechanism which releasably retains the first arm of the elongated body relative to the second arm of the elongated body;
the releasable securement mechanism including a first member on the first arm of the elongated body and a second member on the second arm of the elongated body, the first member and second member releasably engaging in one or more closed positions to retain the first arm of the elongated body relative to the second arm of the elongated body;
a circular aperture located on the elongated body at a position in-between the first and second ends, the aperture having a central axis generally perpendicular to the axis of elongation, and each of the first and second arms of the elongated body defining at least a portion of the circular aperture;
wherein, in at least one closed position, the circular aperture is configured and dimensioned to engage with an outer surface of an endotracheal tube to secure the endotracheal tube relative to the elongated body to prevent movement of the endotracheal tube along the central axis of the aperture.

16. The device of claim 15, wherein the device has an overall arcuate shape.

17. The device of claim 16, wherein the at least one outer surface is generally arcuate along the axis of elongation.

18. The device of claim 15, wherein the at least one outer surface is arcuate to conform to a mouth region of a patient's face.

19. The device of claim 15, wherein the angular curvature of the generally arcuate outer surface is from about 30 degrees to about 1 degree.

20. The device of claim 15, wherein the axis of elongation is from about 3 cm to about 10 cm.

21. The device of claim 20, wherein the axis of elongation is from about 5 cm to about 7 cm.

22. The device of claim 13 wherein a curvature of the generally arcuate outer surface is generally circular and ranges from about 0.04 rad/mm to about 0.001 rad/mm.

23. The device of claim 22 wherein the curvature of the generally arcuate outer surface is from about 0.02 rad/mm to about 0.005 rad/mm.

24. The device of claim 22, wherein a chord of the generally circular curvature is from about 2 cm to about 8 cm.

25. The device of claim 24, wherein an arc length of the generally circular curvature is from about 3 cm to about 10 cm.

26. A method of securing an endotracheal tube to a young patient in need thereof, comprising:
placing an endotracheal tube in the patient;
securing an endotracheal tube securement device according to claim 1 to the endotracheal tube at a position which maintains a desired depth of insertion of the endotracheal tube with the securement device placed against the mouth of the patient; and
securing the endotracheal tube to the mouth of the patient using an adhesive securement means.

27. The method of claim 26, wherein the adhesive securement means is cloth tape.
